(19) United States
(12) Patent Application Publication
(10) Pub. No.: US 2020/0399609 A1
(43) Pub. Date: Dec. 24, 2020

(54) SELF REPLICATING RNA FOR INDUCING SOMATIC DIFFERENTIATION OF UNMODIFIED ADULT STEM CELLS

(71) Applicant: InGeneron, Inc., :Hous, TX (US)

(72) Inventors: Eckhard U. ALT, Houston, TX (US); Tahereh KARIMI, Houston, TX (US); Vladimir N. Potaman, Houston, TX (US)

(21) Appl. No.: 17/008,481

(22) Filed: Aug. 31, 2020

Related U.S. Application Data
(63) Continuation-in-part of application No. 15/590,814, filed on May 9, 2017, now Pat. No. 10,760,059.
(60) Provisional application No. 62/333,845, filed on May 10, 2016.

(51) Int. Cl. C12N 5/0775; C12N 15/86

(52) U.S. Cl. CPC C12N 5/0667; C12N 2506/1307; C12N 2501/60; C12N 15/86

(57) ABSTRACT

A self-replicating RNA for inducing somatic differentiation of unmodified adult stem cells is described. Methods of differentiating unmodified adult stem cells into functional beta-like cells are also described, as well as compositions, tissues and devices containing such cells. The method requires inducing sequential expression of PDX1 before NGN3, and NGN3 before MAFA in these stem cells to form reprogrammed beta-cells. Self-replicating RNAs are provided and introduced into the adult stem cells to induce the sequential expression. Methods of treating diabetes are also provided, comprising obtaining stem cells, preferably from a patient with diabetes, inducing sequential expression of PDX1>NGN3>MAFA, in said stem cells to form reprogrammed beta-cells, and introducing said reprogrammed beta-cells into a pancreas of said patient.

### PRIOR RELATED APPLICATIONS

This application is a continuation-in-part of U.S. Ser. No. 15/590,814, titled REPROGRAMMED BETA-CELLS FROM ADULT STEM CELLS, filed May 9, 2017, which claims priority to U.S. Ser. No. 62/333,845, titled INDUCED BETA-CELLS FROM ADULT STEM CELLS, filed May 10, 2016. Both are incorporated by reference herein in its entirety for all purposes.

### FEDERALLY SPONSORED RESEARCH STATEMENT

Not applicable.

### FIELD OF THE DISCLOSURE

This invention relates to methods for the production of reprogrammed or induced beta-cells for use in the treatment of diabetes, as well as the reprogrammed or induced beta-cells thereby produced and uses for same.

### BACKGROUND OF THE DISCLOSURE

Beta-cells (β cells) are a type of cell found in the pancreatic islets of the pancreas. They make up 65-80% of the cells in the islets. The primary function of a beta-cell is to store and release insulin—a hormone that reduces blood glucose concentration. Beta-cells can respond quickly to spikes in blood glucose concentrations by secreting some of their stored insulin, while simultaneously producing more.

Insulin release is mediated by ion channels. Voltage-gated calcium channels and ATP-sensitive potassium ion channels are embedded in the cell surface membrane of beta-cells. These ATP-sensitive potassium ion channels are normally open and the calcium ion channels are normally closed. Potassium ions diffuse out of the cell, down their concentration gradient, making the inside of the cell more negative with respect to the outside (as potassium ions carry a positive charge). At rest, this creates a potential difference across the cell surface membrane of −70 mV.

When the glucose concentration outside the cell is high, glucose molecules move into the cell by facilitated diffusion, down their concentration gradient through the GLUT2 transporter. Since beta-cells use glucokinase to catalyze the first step of glycolysis, metabolism only occurs around physiological blood glucose levels and above. Metabolism of the glucose produces ATP, which increases the ATP to ADP ratio.

The ATP-sensitive potassium ion channels close when this ratio rises. This means that potassium ions can no longer diffuse out of the cell. As a result, the potential difference across the membrane becomes more positive (as potassium ions accumulate inside the cell). This change in potential difference opens the voltage-gated calcium channels, which allows calcium ions from outside the cell to diffuse into the cell, down their concentration gradient. When the calcium ions enter the cell, they cause vesicles containing insulin to move to, and fuse with, the cell surface membrane, releasing insulin by exocytosis.

Diabetes mellitus is a disease caused by the loss or dysfunction of insulin-producing beta-cells in the pancreas. Specifically, in type 2 diabetes mellitus, beta-cells exhibit an impaired capacity to compensate for increased insulin demand, a defect that has been ascribed to both inadequate cellular capacity to secrete insulin and beta-cell death. In addition, diabetes can be accompanied by peripheral insulin resistance.

This impairment in glucose-stimulated insulin secretion has been attributed to defects in glucose sensing, mitochondrial dysfunction and oxidative stress. Other studies suggest that defects in multiple cellular processes can compromise beta-cell function and can be a factor to induce diabetes mellitus. A simultaneous loss of beta-cell function and identity could be explained by reduced expression of a central transcriptional regulatory network involved in beta-cell differentiation and maintenance. Recent studies suggest that dysregulation of the beta-cell differentiation state is among the earliest events marking the progressive failure of beta-cells in diabetes (FIG. 1).

Therefore, innovative strategies for diabetes therapy aim to replace lost or damaged insulin-producing beta-cells by reprogramming of other cell types towards beta-cell lineage. Previous studies concentrated on the reprogramming of embryonic pluripotent or induced pluripotent stem cells towards beta-cells using various factors (FIG. 2). These differentiated cells, however, are not necessary beta cells and often lack much of the structure and markers that beta-cells need to perform their necessary functions.

Efficient and reproducible differentiation of initially unmodified autologous adult stem cells into glucose-responsive, insulin-producing beta-cells has not yet been fully achieved. Most of the previous studies for production of beta-cells have been based on the attempts to up-regulate one or two insulin inducing factors and had some success, but there is room for further improvement.

Therefore, what is still needed in the art are robust methods for generation of pancreatic beta-cells by genetic re-programming of easily available primary unmodified adult adipose derived stem cells (ADSCs) or other adult stem cells.

### SUMMARY OF THE DISCLOSURE

In one aspect of this disclosure, adult adipose tissue derived stem cells (ADSCs) were reprogrammed towards beta-cell lineages by different combinations of transcription factors including PDX1, NGN3 and MAFA that were sequentially applied to the cells. The sequential induction of transcription factors is accomplished by using self-replicating RNAs (srRNAs) that encode the transcription factors.

In another aspect of this disclosure, a method of inducing stem cells to differentiate into beta-cells is described. The method comprises obtaining adult stem cells from a patient, inducing the sequential expression of PDX1>NGN3>MAFA in the adult stem cells by introducing a self-replicating RNA into the adult stem cells in order to reprogram the adult stem cells, and growing the reprogrammed adult stem cells until reprogrammed beta-cells form (> means before).

In another aspect of this disclosure, a composition comprising a population of reprogrammed beta-cells is described. The population of reprogrammed beta-cells are cells differentiated from adult unmodified stem cells transduced with one or more self-replicating RNAs to allow sequential upregulation of genes encoding PDX1, NGN3, and MAFA, thus forming reprogrammed beta-cells able to produce insulin in response to glucose.

As used herein, PDX1 (pancreatic and duodenal homeobox 1), also known as insulin promoter factor 1, is a transcription factor necessary for pancreatic development, including β-cell maturation, and duodenal differentiation. In humans, this protein is encoded by the PDX1 gene, which was formerly known as IPF1.

As used herein, NGN3, or Neurogenin 3, is another member of the bHLH (basic helix-loop-helix) family of transcription factors. NGN3 functions in the differentiation of endocrine pancreas cells. Although its key function is in the pancreas, intestinal cells and neural cells express NGN3 as well. Several studies have highlighted the importance of NGN3 for differentiation of endocrine cells. In mice, NGN3 is present in cells as the pancreas begins to bud and glucagon cells are formed. There are several pathways that NGN3 works through. NGN3 is a crucial component in pancreatic development and plays a supporting role in intestinal as well as neuronal cell development. Studies have demonstrated that knockout of NGN3 in mice leads to death shortly after birth possibly due to after-effects of severe diabetes.

As used herein, MAFA refers to v-maf avian musculoponeurotic fibrosarcoma oncogene homology A, which is a transcription factor that binds RIPE3b—a conserved enhancer element that regulates pancreatic beta-cell-specific expression of the insulin gene.

As used herein, NKX6.1 or NKX6-1 is also known as NK HOMEOBOX, FAMILY 6, and MEMBER A. In the pancreas, NKX6.1 is required for the development of beta-cells and is a potent bifunctional transcription regulator that binds to AT-rich sequences within the promoter region of target genes.

The upregulation of transcription factors used herein was accomplished through srRNAs. However, this was exemplary only and any expression vector could be used. Alternatively, mRNAs could be used or even intact functional proteins.

DNA, RNA and protein can be introduced into the cells in a variety of ways, including e.g., microinjection, electroporation, and lipid-mediated transfection. RNA can also be delivered to cells using e.g., tat-fusion using e.g., the HIV-1-tat protein. Tat has also been used for protein delivery. For example, a tetramethylrhodamine-labeled dimer of the cell-penetrating peptide TAT, dfTAT, penetrates live cells by escaping from endosomes with high efficiency. Other cell-penetrating peptides (CPPs) are also known, and indeed intact proteins can be delivered using CPPs as fusion proteins, as well as by noncovalent CPP/protein complexes.

At the current time, retroviruses are preferred for gene therapies (retroviral and lentiviral) and have now been used in more than 350 gene-therapy studies. Retroviral vectors are particularly suited for gene-correction of cells due to long-term and stable expression of the transferred transgene(s), and also because little effort is required for their cloning and production. However, retroviral treatment leads to integration of foreign DNA into the host's chromosome, therefore causing safety concerns. There is the need for next generation vectors.

Furthermore, with the advent of genome engineering techniques (such as CRISPR/CAS9 (and the like), it is also possible to selectively activate the needed proteins via genome engineering, rather than by cell delivery of DNA, RNA, or protein. Selective epigenetic changes (e.g., changing methylation patterns) may also be possible in the future.

While a number of different multipotent or pluripotent stem cell types could be used herein, the main value of the invention lies in treating diabetes in humans. Therefore, the preferred source of stem cells are autologous cells, such as e.g., adult adipose tissue derived stem cells (ADSCs). In the future, when umbilical tissue derived stem cells are stored from larger number of donors, e.g., cord blood and umbilical tissue derived stem cells and the like, other types of stem cells in a matched allogenic transplant manner may be preferred, but at the current time, these resources are available for only a few patients.

No one to this point has used adult derived stem cells for the re-creation of beta-cells. Such methods are a tremendous advantage over embryonic stem cells because they can be autologous, eliminating rejection problems, and are readily available, unlike embryonic stem cells. Furthermore, induced stem cells (so-called iPS cells which have de-differentiated to a more stem-like state) may not be safe, as at least one researcher has opined that such cells are very close to cancer cells and our own research confirms this. Adipose tissue is easily accessed with a modicum of discomfort, and many patients have significant amounts of such tissue available for use. Thus, this source is safer and conveniently available in large amounts.

Allogenic cells may also be suitable, although anti-rejection drugs are typically required if the HLA patterns do not match. However, such cells are in use today, and may be more amenable to use in the future as more and more banks collect and store cord blood, cord tissue, etc. and the stem cells generated thereby, particularly where libraries of hundreds and thousands of different HLA patterns can be collected and cryopreserved, so that the probability of a fully matched allogeneic transplant increases.

Alternatively, a library of reprogrammed beta-cells can be generated in advance, so at the time of need, these cells will be readily available for transplantation. One interesting aspect is that in a matched allogenic transplant of reprogrammed beta-cells—despite a full 6 out of 6 match of HLA surface markers—the specific donor cell associated underlying genetics might be different from the recipients genetic pre-disposition to acquire Type 1 Diabetes and therefore the newly generated islet cells from a matched allogenic donor cell sample might not be prone to the autoimmune attack typically present against the host's own beta-cells.

In addition, we have described the invention using human or mouse wild type genes, but other sources may be used as appropriate for the species. Codon optimization can also be performed to optimize expression, and expression vectors or mRNA can also be optimized for use.

The disclosure includes one or more of the following embodiments, in any combination(s) thereof:
A self-replicating RNA (srRNA) for inducing adult somatic stem cells to differentiate into beta-cells, said srRNA comprising a 5′ cap, and sequences encoding nonstructural proteins, a promoter, PDX1, NGN3, MAFA genes, independent ribosome entry sites (IRES), optionally fluorescent marker genes (mCherry or GFP), optionally a selectable marker, and 3′ poly A tail. The srRNA can further include NKX6.1.
A method of inducing adult somatic stem cells to differentiate into beta-cells, said method comprising inducing a sequential expression of PDX1>NGN3>MAFA in a population of adult somatic stem cells by introducing a self-replicating RNA into said adult somatic stem cells in order to reprogram said stem cells, and growing said reprogrammed stem cells until reprogrammed beta-cells form.
A method of inducing stem cells to differentiate into beta-cells, said method comprising obtaining adult stem cells from a patient, inducing the sequential expression of PDX1>NGN3>MAFA in said adult stem cells by introducing a self-replicating RNA into said adult stem cells in order to reprogram said adult stem cells, and growing said reprogrammed adult stem cells until reprogrammed beta-cells form.
Any method herein described, wherein a first srRNA, a second srRNA, and a third srRNA are sequentially transduced into said adult unmodified stem cells, wherein the first srRNA comprises the coding sequence of Pdx1, the second srRNA comprises the coding sequence of Ngn3, and the third srRNA comprises the coding sequence of MafA, and the first srRNA is transduced 1-10 days before transducing the second srRNA, and the second srRNA is transduced 1-10 days before transducing the third srRNA.
Any method herein described, wherein said stem cells are autologous stem cells, preferably autologous adipose derived stem cells, preferably from adult tissue.
Any method herein described, wherein said inducing step uses one or more srRNA encoding PDX1, NGN3, and MAFA, preferably sequentially transducing a single inducible srRNA encoding PDX1, NGN3, and MAFA under protection from interferon-mediated immune response by B18R protein in the conditioned medium that allow a sequential on/off activation of the respective expression of transcription factors.
Any method herein described, wherein said inducing step requires 1 to 6 days of expression of a protein before activating the next protein in the sequence, or more preferably about 3 days.
Any method herein described, wherein said inducing step uses srRNA encoding PDX1, NGN3, and MAFA.
A reprogrammed beta-cell or beta-like cell made by a method described herein and producing insulin in response to glucose.
A composition comprising a population of induced beta-cells differentiated from adult unmodified stem cells transduced with one or more self-replicating RNAs to allow sequential protein upregulation of PDX1>NGN3>MAFA, thus forming reprogrammed beta-cells able to produce insulin in response to glucose. Preferably, the cell population is enriched for the reprogrammed beta-cells before use in patients.
A method of treating diabetes, by treating a patient with any of the reprogrammed beta-cells described herein.
A method of treating diabetes, said method comprising obtaining stem cells from a patient with diabetes, inducing sequential expression of PDX1>NGN3>MAFA proteins in said stem cells by introducing self-replicating RNAs encoding PDX1, NGN3 and MAFA to form reprogrammed beta-cells, and introducing said reprogrammed beta-cells into an artificial pancreas, and surgically placing said artificial pancreas into said patient.
A method of inducing differentiation of mammalian adult unmodified stem cells into somatic cells, comprising: transducing a self-replicating RNA (srRNA) into the adult unmodified stem cells, said srRNA comprising (i) a 5′ cap, (ii) coding sequences of nonstructural proteins, (iii) a promoter, (iv) coding sequences of transcription factors and optionally fluorescent proteins mCherry or GFP, (v) independent ribosome entry sites (IRES), (vi) optionally a puromycin-resistance gene (Puro), and (vii) 3′ poly A tail; and growing said transduced stem cells until differentiated somatic cells form; wherein the transcription factors induce differentiation of the adult unmodified stem cells into somatic cells.

As used herein, “inducing” the expression of certain proteins in stem cells does not imply any particular methodology. Instead, any means of turning on protein expression can be used, including the use of expression vectors, naked DNA or RNA or protein, induced epigenetic changes, and the like. It does not include those natural cells that already demonstrate expression of the recited gene/proteins, but only stem cells that have been re-programmed to do so by the hand-of-man.

By “sequential” expression we mean that the initiation of transcription/translation is staggered, such that one begins before another. However, there may be overlap of transcription/translation times after initiation, e.g., co-expression. Alternatively, a predetermined period may be provided between the expression of each protein, such that sufficient amount of expression of the preceding protein can be achieved for proper reprogramming before initiating the expression of the next protein.

The term “self-replicating RNA” or “srRNA” refers to an RNA that is capable of continuously replicating, as well as transcribing, itself in a host cell as a replicon without the need for a DNA template.

The phrase “a selectable marker” means a marker that can be used to select, positively or negatively, the cells that have been successfully transduced. In one embodiment, the selectable marker is a puromycin-resistance gene (Puro), but other selectable marker can be used.

In our experimental method, the expression order of proteins (including PDX-1, NGN-3 and MAFA) was found to be important. Therefore, in our experimental design, we applied three different srRNAs, one for each protein, for sequential expression of the three proteins. In our experimental design, modeled from the development of pancreatic β-cells at the early stages of embryogenesis, there was little or no overlap in expression between the three proteins. However, there remains the possibility of differentiation of stem cells to pancreatic beta-cells (with lower efficiency of differentiation and maturation) with some degree of co-expression of PDX-1, NGN-3 and MAFA.

As used herein, “stem cells” include a totipotent, pluripotent and multipotent stem cells. These are cells that are not yet differentiated, and thus can either form cells of all three germ layers and cell types, or a multiplicity of cell types upon receiving the appropriate signaling from its microenvironment. These cells can be isolated by appropriate means from the blood vessels of all tissues and organs.

As stem cells are small, early in their development stage, and are initially silenced cells, the majority of the scientific community characterizes stem cells incorrectly. These early cells do not express the surface markers typically believed to characterize stem cells such as CD34, CD44, CD90, or CD105 as reported in the literature. Instead, these stem cells express CD29, CD49, Nestin, Oct 4, Sca-1 and SSEA3/4 and markers of ABC cassette pumps such as ABCB5 and other early embryonic markers, without reprogramming of these cells to embryonic cells or making them related to the “cancer risk” iPS cells.

“Adult unmodified stem cells” are typically multipotent stem cells that are derived from a non-infant person and have not been genetically modified. The term does not imply any particular age. Also known as somatic stem cells, they can be found in children, as well as adults.

As used herein, “reprogrammed beta-cells” or “beta-like cells” or “reprogrammed beta-like cells” are used interchangeably. These are cells that used to be to be naturally (not induced) toti-, multi-, or pluri-potent, but have been intentionally differentiated or “reprogrammed” by the methods described herein, and thus demonstrate the defining characteristic of being able to correctly secrete insulin, but not glucagon, in response to high glucose. The term does not include prior art partially differentiated cells that demonstrate incorrect regulation of insulin secretion, such as those described by Xu (2013) and Akinci (2013), or that co-secrete glucagon in response to glucose.

The reprogrammed beta-cells may differ in other respects from wild type beta-cells however, reflecting both the transfected genes as well as possible imperfections in the differentiation process.

As used herein, “autologous” means cells derived from the patient. “Allogenic” refers to cells derived from the same species but having a different genotype. “Syngeneic” refers to cells from a genetically identical source, such as a twin, hence immunologically compatible or so closely related that transplantation does not provoke an immune response.

The “artificial pancreas” is a technology in development to help people with diabetes automatically control their blood glucose level by providing the substitute endocrine functionality of a healthy pancreas. Different approaches under consideration include:

1) The medical equipment approach—using an insulin pump under closed loop control using real-time data from a continuous blood glucose sensor.

2) The bioengineering approach—the development of a bio-artificial pancreas consisting of a biocompatible sheet of encapsulated beta-cells. When surgically implanted, the islet sheet will behave as the endocrine pancreas and will be viable for years.

3) The gene therapy approach—the therapeutic infection of a diabetic person by a genetically engineered virus, which causes a DNA change of intestinal cells to become insulin-producing cells.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed and excludes all additional elements.

The phrase “consisting essentially of” excludes additional material elements but allows the inclusions of non-material elements that do not substantially change the nature of the invention.

The following abbreviations are used herein:

| ABBREVIATION | TERM |
|---|---|
| αMEM | MEM with Eagle's balanced salts |
| ADST | Adipose Tissue derived stem cells |
| cDNA | Copy DNA |
| DNA | Deoxyribonucleic acid |
| FBS | Fetal bovine serum |
| MEM | Minimum-essential medium |
| PBS | Phosphate buffered saline |
| PCR | Polymerase chain reaction |
| qPCR | Quantitative PCR |
| RNA | Ribonucleic acid |
| mRNA | Messenger RNA |
| srRNA | Self-replicating RNA |
| VEE | Venezuelan equine encephalitis virus |

The following genes/proteins are discussed herein:

FAS: Fas Cell Surface Death Receptor, Previous symbol name: TNF
Receptor Superfamily, Member 6 FAS (NM 000043)
FGF-10: Fibroblast Growth Factor-2
FGF-2: Fibroblast Growth Factor-2
FLIP: FLICE-like inhibitory protein, aka c-FLIP, now called CFLAR
or CASP8 and FADD-like apoptosis regulator.
Fox- A1 and A2: Forhead Box a1 and a2
IL1B: interleukin 1 beta
ISL-1: ISELT-1 or ISL LIM homeobox 1
MAFA: v-maf avian musculoponeurotic fibrosarcoma oncogene
homology A
Neuro D: Neuronal differentiation 1
NGN3: Neurogenin-1
NKX6.1: Nkx 6 homebox1
PAX4, 6: Paired Box4
PDX1: Pancreatic and duodenal homebox-1
Ptf1a: Pancreatic Specific transcription factor- 1A
SHH: Sonic hedgehog
CD29, Integrin beta-1 also known as CD29 is a protein that in humans
is encoded by the ITGB1 gene
CD49, CD49a is an integrin alpha subunit.
Nestin, a type VI intermediate filament (IF) protein.
Sca-1 “Stem cells antigen-1”. A common biological marker used to
identify hematopoitic stem cell (HSC) along with other markers.
Oct-4, (octamer-binding transcription factor 4) also known as (POU
domain, class 5, transcription factor 1) is a protein that in humans
is encoded by the POU5F1 gene
SSEA3/4 Cell Surface Glycosphingolipids SSEA-3 and SSEA-4
ABCB5 belongs to the ATP-binding cassette (ABC) transporter
superfamily of integral membrane proteins. ATP-binding cassette
sub-family B member 5 also known as P-glycoprotein ABCB5 is a
plasma membrane-spanning protein that in humans is encoded
by the ABCB5 gene.
nsP1-nsP4: nonstructural proteins 1 to 4 from Venezuelan equine
encephalitis virus.

### DESCRIPTION OF FIGURES

FIG. 1 Hypothetical model illustrating the consequence of hyperglycemia on β-cell production of IL-1β in parallel with insulin secretion. The paracrine effect of IL-1β induces FAS engagement, which in the presence of c-FLIP leads to β-cell proliferation, differentiation, and increased function. From Donath 2005.

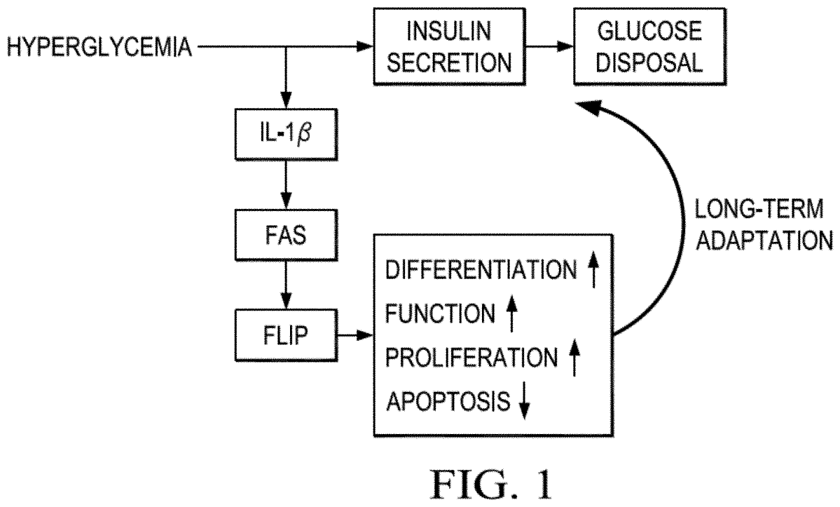

FIG. 2 Schematic representative of sequence of events and transcription factors involved in development of pancreatic beta-cells. According to our findings, the molecular transcription factors defined in green color, are the critical ones for the sequential induction of differentiation towards the insulin producing cells and differentiate from the other factors, such as ISL-1, that are existing in other developmental pathways and are not specific and critical for the pancreatic lineage commitment. LEGEND: Shh: Sonic hedgehog; Fox-a1 and a2: Forkhead Box a1 and a2; FGF-2: Fibroblast Growth Factor-2; FGF-10: Fibroblast Growth Factor-10; Pdx-1: Pancreatic and duodenal homebox-1; Ptf1a: Pancreatic Specific transcription factor-1A; ISL-1: ISLET-1 or ISL LIM homeobox 1; Ngn-3: Neurogenin-1; Neuro D: Neuronal differentiation 1: Mafa: v maf avian musculoaponeurotic fibrosarcoma oncogene homology A: Pax4, 6: Paired Box4; Nkx 6.1: Nkx 6 homebox-1

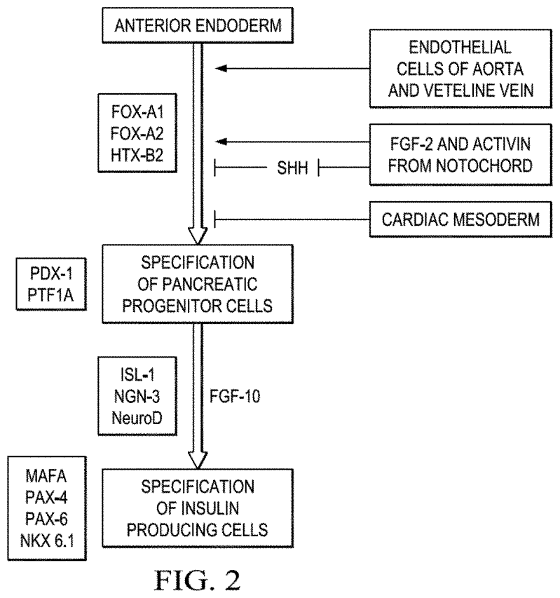

FIG. 3 Time course of transductions and sequential expression used in our experiments.

FIG. 4 Schematic of method of preparing ADSCs.

FIG. 5. Multi-step construction of srRNA expression vector of this disclosure.

FIG. 6. Schematic of using srRNA to differentiate ADSCs into beta-cells.

FIG. 7A-B. Exemplary time courses of sequentially transducing srRNAs to induce differentiation of adult unmodified stem cells into beta cells.

### DETAILED DESCRIPTION

To differentiate ADSCs into pancreatic islet cells, adipose tissue derived stem cells are transduced for sequential transcription and translation, with different combinations of β-cell inducing factors, including PDX1, NGN3 and MAFA, such that the proteins appear in the differentiating cell sequentially (PDX1>NGN3>MAFA).

Although we used sequential transduction to demonstrate proof of concept, some overlap in the time frames of action of transcription factors may be preferred or acceptable, which would of course require co-transfection of srRNAs or using sRNAs encoding at least two transcription factors. However, this will subject the cells to less stress during the transduction or transfections and may be preferred. In yet another embodiment, the proteins themselves could be introduced sequentially, or mRNA encoding same.

Akinci (2013) attempted a similar experiment and did induce some differentiation towards beta-cells. However, he did not use adult human stem cells but rather a rat pancreatic exocrine cell line (AR42J-B13). In addition, this group transfected all three genes at the same time and not sequentially, as described herein. They were thus unable to achieve glucose sensitive secretion of insulin, although they could at least induce insulin production.

Oh (2015) used exosomes from insulinoma derived cell lines to induce differentiation of bone marrow cells. However, insulinoma is a semi-malignant form of a pancreatic tumor, and therefore presents risk that not only the features of the beta-cells are brought forward by the exosomes, but that they also could generate new insulinomas. Our own experiments confirm that the addition of exosomes from breast cancer cells to normal adipose derived stem cells significantly changes their gene expression profile and their features in direction of invasiveness and tumor formation. Thus, the use of exosomes from insulinoma derived cell lines is clinically unacceptable for safety reasons.

Millman (2015) used patient-derived human induced pluripotent stem cells from skin fibroblasts using sequential use of various factors (different from those discussed herein) in the media. The resulting cells were able to secrete insulin in response to glucose, and few cells expressed the α-cell hormone glucagon. However, as noted herein, induced pluripotent cells are believed to have evolved in the direction of tumorigenesis, and thus present significant safety risks.

Lima (2016) tested a variety of transcription factors, including PDX1, NGN3, MAFA and PAX4, added at the same time to exocrine tissue cells isolated from the pancreas of brain-dead donors. Lima found that most efficient transcription factor combination for the ex vivo reprogramming of exocrine pancreatic cells towards β-cells resulted from the concerted actions of PDX1, NGN3, MAFA and PAX4. Inclusion of PAX4 appeared to be crucial for generating glucose responsive beta-like cells. Those cells, however, expressed insulin at about 15-30% of the levels in human islets. Inclusion of late stage inhibition of ARX using siRNA significantly decreased glucagon mRNA and protein levels, making these reprogrammed cells promising. However, such cells were not autologous, and thus rejection remains problematic.

Xu (2013) was able to demonstrate that the co-expression of PDX1 and MAFA during a specific time window of development can act synergistically with either NGN3 or NEUROD to promote the differentiation of mouse embryonic stem cells into insulin-secreting cells. This group showed co-expression of PDX1 and MAFA with either NGN3 or NEUROD at the final stage of a three-step differentiation process, significantly increased the differentiation efficiency. It also increased the glucose-stimulated insulin and C-peptide secretion in insulin-secreting cells derived from mouse embryonic stem cells (mES cells) compared to the control green fluorescent protein (GFP) vector-transduced group. Unfortunately, neither embryonic stem cells nor induced pluripotent cells (iPS) can be used for clinical applications due to the existing risk of tumorigenesis.

Further, although staging the differentiation process, the Xu group still used co-expression of the above factors, not sequential expression. Thus, mES cells were first induced to make embryoid bodies for 48 hours, then stimulated using activin A for 2 days. Second, those cells were expanded using fibroblast growth factor and epidermal growth factor for 5 days. Third, the cells were then matured and the differentiation factors adding alone, in pairs, in triplet combinations and all four at once for another 5 days. The two factor groups increased insulin 1 levels by 3-fold, but the three factor groups increased expression 15-fold. No further increase was observed with the fourth factor.

Generally speaking, we used adipose tissue derived stem cells (ADSCs) transfected or transduced with srRNAs encoding PDX1, NGN3 and MAFA, such that each of the proteins was sequentially expressed. However, the adult adipose derived stem cells are exemplary only and suitable pluripotent adult stem cells recovered from other sources can be used as well. There can be a predetermined gap between each transduction.

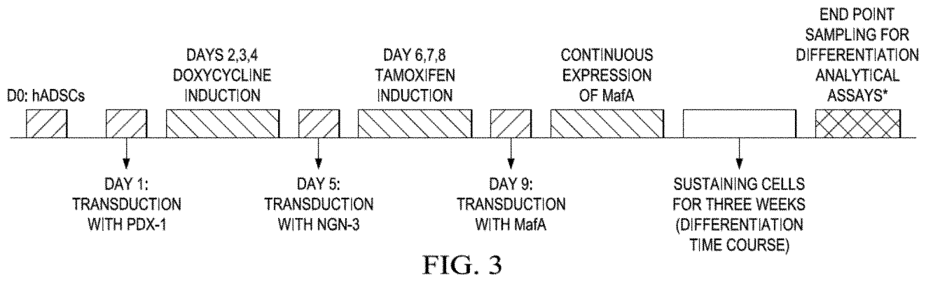

In our experience, the proteins needed to be sequentially introduced into the cell in a particular sequence order and amount (FIG. 3). Attempting to express all genes at once or not keeping the correct sequence of expression can lead to unsatisfactory results, thus in proof of concept work, we performed sequential transductions.

It has been proposed to perform cell programming by synthetic mRNA, including derivation of induced pluripotent stem cells from differentiated cells, differentiation of stem cells into osteogenic, pancreatic and neuronal lineages, as well as trans-differentiation between cell lineages. The mRNA templates transduced into cells contain all elements necessary for protein translation: the 5′ cap, 5′ and 3′ untranslated regions (UTR), the gene-coding region and poly-A sequences. The 5′ cap and 3′ poly-A binding proteins form a complex and bring the RNA ends together to form a closed-loop mRNA template, where the ribosomes circulate and efficiently translate the protein. Although the mRNA ends in the circle are protected from exo-ribonuclease binding, the mRNA is still degraded by other enzymes on a timescale of several hours. Therefore, protein translation in the cells from synthetic mRNA requires its repeated transduction as frequently as once or twice per day.

To have sustaining reprogramming effect on the adult stem cells, a self-replicating RNA (srRNA) approach is described. RNA-containing viruses, such as Venezuelan equine encephalitis (VEE) virus, contain a positive-sense single-stranded RNA that encodes four nonstructural proteins (nsP1 to nsP4). These nonstructural proteins are translated directly from genomic RNA, and together with cellular proteins they form an RNA-dependent RNA polymerase complex needed for viral genome replication without DNA intermediates and for transcription of the downstream RNA sequence encoding viral structural proteins. Thus, the VEE viral RNA genome contains all necessary elements for self-replication and self-transcription. It was also shown that the sequences encoding viral structural proteins may be replaced with those encoding the desired transgenes. As a result, the expression of non-infectious viral genome would generate transgenic proteins rather than those needed for the functional VEE virus.

The capacity of VEE viral genome is 8.3 kb, enough to accommodate several transgenes arranged in a polycistronic manner. For example, consecutive transgene-coding sequences may be separated by 2A ribosome skip sequences or by independent ribosome entry sites (IRES). Currently available versions of SP6 and T7 RNA polymerases allow efficient in vitro RNA synthesis of up to 25 kb in length, such that the entire self-replicating RNA (srRNA) genome may be generated in vitro. Then, the srRNA may be delivered into cells by conventional transduction methods. Such srRNA expressing four transcription factors has been tested for reprogramming into induced pluripotent stem cells (iPSC) of fibroblasts and renal epithelial cells.

To select the transduced cells, each srRNA can optionally contain the sequence encoding the fluorescent protein marker (either mCherry or GFP). These markers allow quantification of transduction rates, convenient cell monitoring during culture and FACS sorting of transduced cells. For an easy positive selection of transduced cells, the puromycin-resistance gene was included in the transgene cassette.

To counteract a strong interferon-mediated cellular response to transduced RNA, the interferon I-neutralizing protein B18R from Western vaccinia virus was employed either as a protein expressed from a separate mRNA or as a component of conditioned medium. Earlier, it was shown that a single application of srRNA enabled an extended duration of transgene expression from srRNA that persisted at the level of 10-20% that observed initially after transduction. A comparison of synthetic mRNA and srRNA-based reprogramming showed that a single transduction of srRNA resulted in better efficiency of iPSC reprogramming from somatic cells than a daily mRNA transfection for two weeks.

The experimental system used in this disclosure is described below. To prepare the srRNAs, the original srRNA production vector Simplicon-E3L purchased from Millipore-Sigma contained the promoter for T7 RNA polymerase followed by coding sequences for four non-structural proteins, cloning sites for transgenes, and coding sequences for E3L (a Vaccinia virus protein affecting interferon signaling pathways) and puromycin-resistance gene Puro (for positive selection of treated cells). To reduce the cellular immune response and stabilize srRNA after its transduction into cells, the E3L protein generated from the same RNA and the separately applied B18R protein are used. B18R is an IFN decoy receptor for type I IFNs encoded by the Vaccinia virus genome and by the genomes of other orthopoxviruses. B18R binds to type I IFN from multiple species and prevents IFN signaling through its receptors.

Such a design does not seem optimal for transient expression of the beta-cell differentiation transcription factors—the expression of E3L may keep the presence of transgenes past the time they were needed. For this reason, the commercial vector has been modified to delete the E3L-coding sequence in the second vector line. As a result, the immune response should be offset by the B18R protein to be made from a second mRNA or applied to cells as a part of conditioned medium.

Two sets of srRNA vectors were created in this disclosure. In one set, each srRNA encodes one transcription factor, whereas in the second set, srRNAs contain pairs of protein-coding sequences: Pdx1-Nkx6.1 and Ngn3-MafA. The transgenes to differentiate ADSCs to beta-cells were cloned upstream of the fluorescent markers and Puro-coding sequences. Such srRNA may express the desired transgenes, the affected cells may be monitored by fluorescent markers, selected from the population of transduced and non-transduced cells by FACS or due to expression of the Puro gene, and the proteins may be expressed from srRNA as long as B18R protein counteracts the cellular immune response against viral RNA.

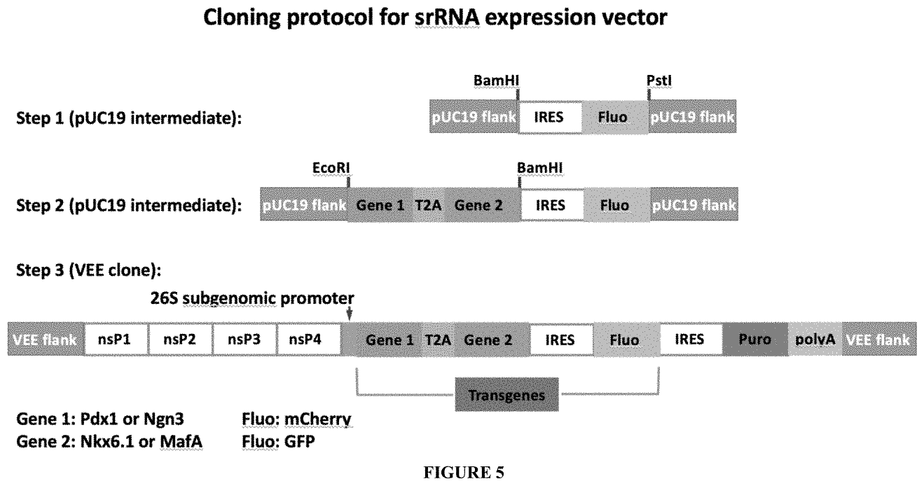

The transgenes were cloned in a multi-step approach, as shown in FIG. 5. In step 1, the IRES-fluorescent marker coding sequence generated by PCR from the lentiviral constructs including unrelated transgenes and IRES-mCherry or IRES-GFP sequences were inserted by ligation into an intermediate DNA cloning vector pUC19 between the BamHI and PstI sites. FIG. 5 shows an IRES-GFP sequence being inserted.

In step 2, the confirmed pUC19 clones with mCherry and GFP were opened up between the EcoRI and BamHI sites. Four human transgenes (PDX1, NGN3, MAFA and NKX6.1) were inserted separately and pairwise by the recombination-based HiFi assembly. As a result, four pUC19 intermediates contained single transcription factor genes, one intermediate pUC19 construct carried the PDX1-T2A-NKX6.1-IRES-mCherry insert, and the other intermediate construct carried the NGN3-T2A-MAFA-IRES-GFP insert. T2A refers to ribosome skip sequence.

In Step 3, the inserts were excised from pUC19 intermediates at the NruI and HpaI restriction sites, then inserted into a VEE vector by the HiFi assembly between the NdeI and NotI sites.

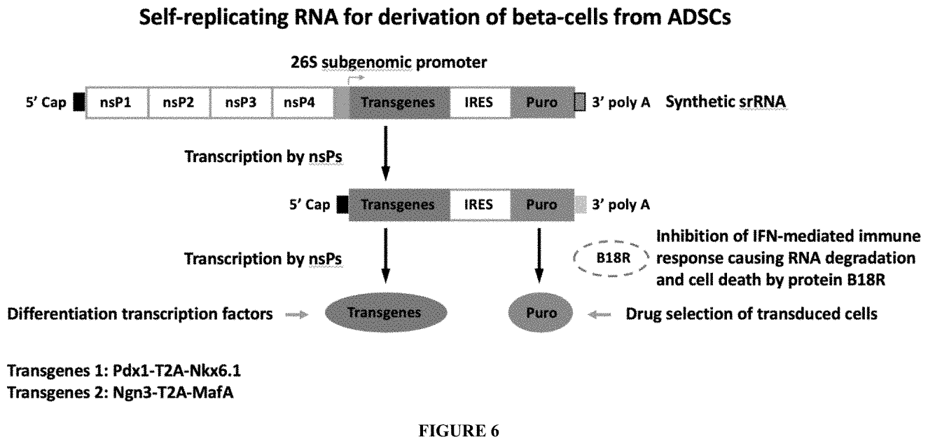

FIG. 6 shows that the resultant DNA constructs contained the service elements (T7 promoter for RNA polymerase to synthesize the entire srRNA, coding sequences for non-structural proteins nsP1 to nsP4, and 26S subgenomic promoter for nsPs to synthesize the desired transgenes) and the transgenic parts (Set 1: PDX1-IRES-mCherry-IRES-Puro; NKX6.1-IRES-GFP-IRES-Puro; NGN3-IRES-mCherry-IRES-Puro; MAFA-IRES-GFP-IRES-Puro; Set 2: PDX1-T2A-NKX6.1-IRES-mCherry-IRES-Puro and NGN3-T2A-MAFA-IRES-GFP-IRES-Puro). The srRNA plasmids after cloning were subjected to diagnostic restriction digestions with several enzymes (AfiII, KpnI, MluI, NdeI, NotI, SphI) and DNA fragments of expected sizes were observed in agarose gel, which confirmed the presence of all transgenes. The entire cloned inserts in plasmids were then sequenced to confirm the absence of mutations.

The transduction protocol includes ADSC plating in serum-free DMEM, adding the srRNA and B18R mRNA pre-mixed with the MessengerMax Transfection Reagent (ThermoFisher) and incubation at 37° C. for 4 hours in a CO₂ incubator before the media change for differentiation. The srRNA expression is checked one day after transduction using flow analysis. The presence of mCherry and GFP coding sequences in the RNA template results in red or green fluorescence in the transfected cells.

The expression of the Puro gene supports positive selection of transduced cells. The B18R mRNA delivery into cells to counteract their immune response initially serves its purpose, this mRNA is not self-replicating and application of more B18R is needed during extended culturing. For this, the B18R-conditioned medium is a cost-effective source of the protein. The B18R-E3L vector is transfected into human foreskin fibroblasts and cultured for 24 hours after which the culture medium is collected. The DNA template is transcribed into B18R and E3L RNA and then proteins.

B18R is a secretory protein and therefore it is secreted into the culture medium after translation but E3L is a cytoplasmic protein which stays in fibroblast cells. Thus, the conditioned medium containing B18R is collected, filter-sterilized and used at 10-20% dilution for culturing ADSCs during their differentiation into beta-cells. A change to medium without B18R re-activates the cellular immune response which leads to srRNA degradation and cessation of induced transcription factor effects.

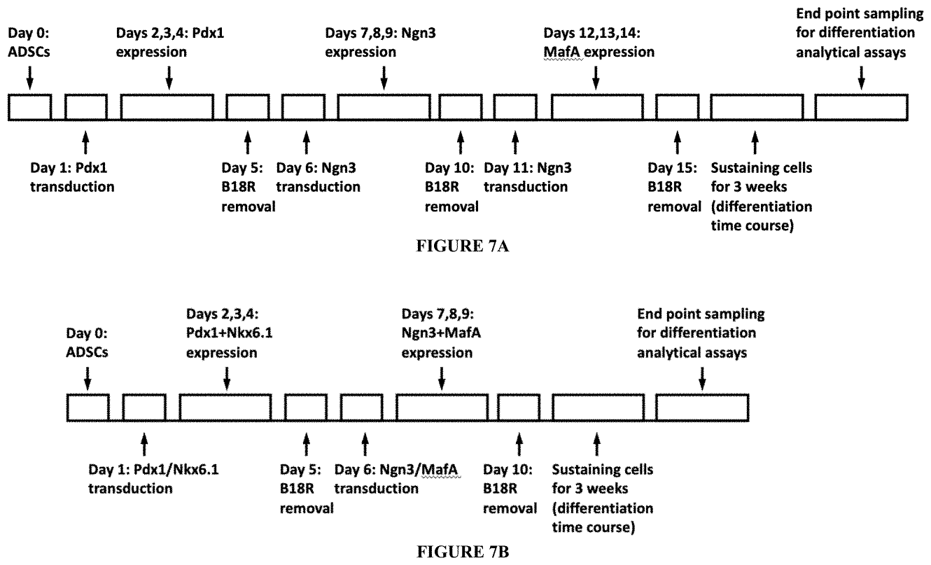

FIG. 7 shows an exemplary time course of sequentially transducing srRNAs into the ADSCs to induce differentiation. As shown in FIG. 7A, on day 1, the first srRNA only encoding Pdx1 is transduced into the ADSCs, followed by three days of PDX1 expression. At this time the medium is supplemented with B18R. On day 5, the B18R supplement is removed. One day of cell culture in the medium lacking B18R is sufficient for srRNA to be degraded and cease to express the encoded transcription factor. The absence of fluorescent signal elicited by a marker protein (mCherry or GFP) will confirm the srRNA degradation. Therefore, on day 6 the second srRNA encoding Ngn3 is transduced into the ADSCs with B18R supplement as described above, followed by three days of NGN3 expression. It is preferred that this srRNA also encoded a different fluorescent marker than the preceding one to clearly see the different transduction and degradation thereof. On day 10, B18R is again removed from the medium to allow degradation of the second srRNA. On day 11, the third srRNA encoding MafA is transduced into the ADSCs with B18R supplement, followed by three days of MAFA expression. On day 15, B18R supplement is removed from the medium, and the cells are allowed to grow for three weeks for the differentiation to take place. In such a way, all necessary srRNAs are sequentially transduced and eliminated from the differentiation protocol. It is noted that the interval between each transduction may be varied, and can range between 1 and 10 days.

To test possible effects of two simultaneously present transcription factors on beta-cell differentiation as indicated by Xu (2013) and Ida (2018), srRNAs encoding two-factor combinations PDX1-NKX6.1 and NGN3-MAFA are sequentially transduced into ADSCs as described above, as shown in FIG. 7B. These srRNAs contain different fluorescent markers to ensure that the time overlap in protein expression is reliably controlled.

The srRNAs are expected to induce differentiation of ADSCs into beta cells, with the sequential expression discussed above.

Although our experiments used 3 or more days before initiating the next differentiation factor, this length of time can vary (e.g., 1-10 days), possibly being reduced in healthier cells or where the differentiation factors have very strong promoters and well optimized expression vectors or other vehicles. By contrast, where the cells are weaker or are recovering from the shock of a transfection or transduction procedure, or where the expression vectors are not optimized, then longer periods may be needed. Thus, the time period can vary from 12 hour to 10 days or more, preferably at least 24, 36, 48, 72, 96 or 120 hours or more. We also contemplate that our results can be improved by optimizing the expression time for each gene for optimal differentiation results.

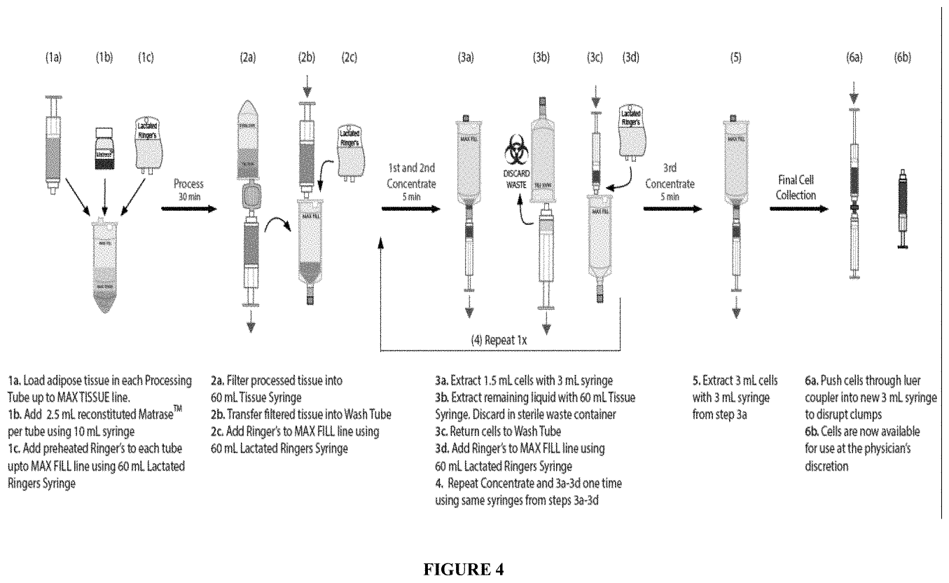

To prepare ADSCs, human subcutaneous adipose tissue was obtained from patients undergoing elective lipoaspiration with informed consent and ADSCs produced by a protocol as shown in FIG. 4 (Winnier et al. 2019). 1a. Load adipose tissue in each Processing Tube up to MAX TISSUE line. 1b. Add 2.5 mL reconstituted Matrase™ per tube using 10 mL syringe. 1c. Add preheated Ringer's solution to each tube up to MAX FILL line using 60 mL Lactated Ringers Syringe. 2a. Filter processed tissue into 60 mL Tissue Syringe. 2b. Transfer filtered tissue into Wash Tube. 2c. Add Ringer's solution to MAX FILL line using 60 mL Lactated Ringers Syringe. 3a. Extract 1.5 mL Cells with 3 mL syringe. 3b. Extract remaining liquid with 60 mL Tissue Syringe. Discard in sterile waste container. 3c. Return cells to Wash Tube. 3d. Add Ringer's solution to MAX FILL line using 60 mL Lactated Ringers Syringe. 4. Repeat Concentrate and 3a-3d one time using same syringes from steps 3a-3d. 5. Extract 3 mL cells with 3 mL syringe from step 3a. 6a. Push cells through luer coupler into new 3 mL syringe to disrupt clumps. 6b. Cells are now available for use at the physician's discretion.

In brief, adipose tissue was washed thoroughly, minced, and incubated with Ringers lactate containing a combination of collagenase I and II and a neutral protease (Matrase™ Reagent, InGeneron Inc., Houston Tex.) in a Tissue Processing Unit (Transpose RT™ System, InGeneron Inc.) for 30 minutes at 40° C. Subsequently, the cell suspension was filtered through a 100-μm filter, washed twice, and then centrifuged at 600 rpm for 5 minutes. The adipose stromal vascular fraction was resuspended in αMEM with 20% FBS, L-glutamine, and penicillin-streptomycin-amphotericin B (SigmaAldrich®) at 37° C. in 5% CO₂. Red blood cells in the supernatant and nonadherent cells were removed after 48 hours. For all experiments shown, human subcutaneous adipose tissue-derived cells were used prior to passage 6.

If desired, ADSCs can be further enriched using ADSC specific markers. The separation of adipocyte precursor populations from nonadipogenic cells using a single cell surface marker is almost impossible, but with the help of multicolor flow cytometry, these putative progenitor cells can be differentiated from nonadipogenic cells such as endothelial and blood cells and thereby enriched. The markers include CD29, CD49, Nestin, Oct 4, Sca-1 and SSEA3/4 and markers of ABC cassette pumps such as ABCB5. Beta-like cells can also be enriched after reprogramming in a similar manner, but using beta-cell specific markers, and/or can be further amplified in culture.

Future work will include confirmatory experiments to demonstrate accurate response to glucose using patch clamp experiments. Patch-clamp recordings of srRNA transfected cells are performed according to a routine protocol. The extracellular solution contains Na-acetate (140 mM), CaCl₂ (1 mM), MgCl₂ (1 mM), HEPES (10 mM) (pH 7.4, adjusted with NaOH) and TTX 0.5 mM (blocks the sodium channel), and nisoldipine 200 nM (eliminates the L-type calcium current). Glucose 20 mM will be added to the superfusing solution.

Future work will also include rescuing diabetic mice models with the newly reprogrammed beta-like cells. Six- to eight-week-old BALB/c transgenic mice that modified for conditional expression of luciferase under the control of the Insulin-1 gene, are made hyperglycemic by i.p. injection of streptozotocin (STZ; Sigma) at 220 mg/kg of body weight. When blood glucose reach levels >16.7 mmol/L and are maintained stably for 1 week, mice are transplanted with 2×10⁶ of the reprogrammed pancreatic beta-cells in 0.1 ml PBS under the renal capsule.

Blood glucose levels will be monitored twice a week in samples obtained from the tail vein of mice by using Accutrend strips (Roche Diagnostics, Indianapolis, Ind.). Grafts are removed after 14 days, and analyzed by immune-histochemistry for the presence of insulin producing cells. Mice are monitored 1 day later for changes in blood glucose levels. Serum is collected from the orbital plexus of mice for human C-peptide levels analysis. The ultrasensitive human C-peptide ELISA kit (Mercodia) with 3% cross reactivity to proinsulin but no cross reactivity to mouse C-peptide and mouse insulin ELISA kit is used according to the manufacturer's instruction.

For intraperitoneal glucose tolerance test (IPGTT), normal non-diabetic mice (n=4) and diabetic mice (n=4) with normalized glucose levels following the transgenic cell transplantation are fasted for 6 h and then given an i.p. injection of glucose (2 g/kg of body weight). Blood glucose is monitored at 0, 30, 60, 90, and 120 min after the glucose injection.

Once the mice experiments confirm safety and efficacy, trials can be initiated in humans, but this is expected to require 2-5 years of additional work.

Each of the following references is incorporated by reference herein in its entirety for all purposes:
Akinci E, et al. (2013) Reprogramming of pancreatic exocrine cells towards a beta (β) cell character using Pdx1, Ngn3 and MafA. Biochem J 442: 539-550
Badieyan Z S & Evans T (2019) Concise review: Application of chemically modified mRNA in cell fate conversion and tissue engineering. Stem Cells Transl Med 8: 833-843.
Cavelti-Weder C, et al. (2018) Reprogramming of pancreatic acinar cells to functional beta-cells by in vivo transduction of a polycistronic construct containing Pdx1, Ngn3, MafA in mice. Curr Protoc Stem Cell Biol 40: 4A.10.1-4A.10.12.
Donath M Y, et al. (2005) Mechanisms of β-cell death in Type 2 Diabetes. Diabetes 54: S108-S113.
Drews K, et al. (2012) The cytotoxic and immunogenic hurdles associated with non-viral mRNA-mediated reprogramming of human fibroblasts. Biomaterials 33: 4059-4068.
Gefen-Halevi S, et al. (2010) Nkx 6.1 Promotes PDX1 Induced Liver To Pancreatic B-Cells Programming. Cellular Reprogramming. 12: 655-664.
Gittes G K (2009) Developmental Biology of the Pancreas: A comprehensive review, Dev Biol 326: 4-35.
Greggio C (2013) Artificial three-dimensional niches deconstruct pancreas development in vitro. Development 140: 4452-4462.
Ida H, et al. (2018) Establishment of a rapid and footprint-free protocol for differentiation of human embryonic stem cells into pancreatic endocrine cells with synthetic mRNAs encoding transcription factors. Stem Cell Res Ther 9: 277.
Kroon E, et al. (2008) Pancreatic endoderm derived from human embryonic stem cells generates glucose-responsive insulin secreting cells in vivo. Nat. Biotechnol 2008: 443-452.
Lima M J, et al. (2016) Generation of functional beta-like cells from human exocrine pancreas. PLoS ONE 11(5): e0156204.
Millman J R, et al. (2016) Generation of stem cell-derived b-cells from patients with type 1 diabetes. Nat Comm 7: 11463.
Morgan R A, et al. (2017) Hematopoietic stem cell gene therapy: progress and lessons learned. Cell Stem Cell 21: 574-590.
Oh K, et al. (2015) In vivo differentiation of therapeutic insulin-producing cells from bone marrow cells via extracellular vesicle-mimetic nanovesicles, ACS Nano 9: 11718-11727.
Petrakova O, et al. (2005) Noncytopathic replication of Venezuelan equine encephalitis virus and eastern equine encephalitis virus replicons in mammalian cells. J Virol 79: 7597-7608.
Sahin U, et al. (2014) mRNA-based therapeutics—developing a new class of drugs. Nat Rev Drug Discovery 13: 759-780.
Saxena P, et al. (2017) Generation of glucose-sensitive insulin-secreting beta-like cells from human embryonic stem cells by incorporating a synthetic lineage-control network. J Biotechnol 259: 39-45.
Schaffer A E, et al. (2013) Nkx 6.1 controls a gene regulatory network required for establishing and maintaining pancreatic beta cell identity. PLOS Genetics 9: 1-15.
Steinle H, et al. (2017) Reprogramming of urine-derived renal epithelial cells into iPSCs using srRNA and consecutive differentiation into beating cardiomyocytes. Mol Ther: Nucleic Acids 17: 907-921.
Steinle H, et al. (2019) Generation of iPSCs by nonimtegrative RNA-based reprogramming techniques: benefits of self-replicating RNA versus synthetic mRNA. Stem Cell Intl, article ID 7641767
Warren L & Lin C (2019) mRNA-based genetic reprogramming. Mol Ther 27: 729-734.
Winnier G E, et al. (2019) Isolation of adipose tissue derived regenerative cells from human subcutaneous tissue with or without the use of an enzymatic reagent. PLoS ONE 14(9): e0221457.
Xu H, et al. (2013) The combined expression of Pdx1 and MafA with either Ngn3 or NeuroD improves the differentiation efficiency of mouse embryonic stem cells into insulin-producing cells. Cell Transplant 22: 147-158.
Yisraeli J K & Melton D A (1989) Synthesis of long, capped transcripts in vitro by SP6 and T7 RNA polymerases. Meth Enzymol 180: 42-50.
Yoshioka N, et al. (2013) Efficient generation of human iPSCs by a synthetic self-replicative RNA. Cell Stem Cell 13: 246-254
Zhu Q, et al. (2008) In vivo reprogramming of adult pancreatic exocrine cells to b-cells. Nature 455: 627-633.

What is claimed is:

1. A self-replicating RNA (srRNA) for inducing adult somatic stem cells to differentiate into beta-cells, said srRNA comprising (a) a 5′ cap, (b) coding sequences of nonstructural proteins, (c) a promoter, (d) coding sequences of at least one of pancreatic and duodenal homeobox 1 (“Pdx1”), Neurogenin 3 (“Ngn3”), v-maf avian musculoponeurotic fibrosarcoma oncogene homology A (“MafA”) and optionally a coding sequence for a fluorescent protein, (e) an independent ribosome entry site (IRES), (f) optionally a selectable marker, and (g) 3′ poly A tail.

2. The self-replicating RNA of claim 1, wherein the nonstructural proteins are nsP1, nsP2, nsP3 and nsP4 of Venezuelan equine encephalitis (VEE) virus.

3. The self-replicating RNA of claim 2, wherein the promoter is a 26S subgenomic promoter.

4. The self-replicating RNA of claim 1, further comprising coding sequence of Nkx6.1.

5. A method of inducing adult somatic stem cells to differentiate into beta-cells, said method comprising:
a) inducing a sequential expression of PDX1 before NGN3, and NGN3 before MAFA in a population of adult unmodified stem cells by transducing a self-replicating RNA of claim 1 into said adult somatic stem cells in order to reprogram said stem cells, and
b) growing said reprogrammed stem cells until reprogrammed beta-cells form.

6. The method of claim 5, wherein a first srRNA encoding PDX1, a second srRNA encoding NGN3 and a third srRNA encoding MAFA are sequentially transduced into the adult unmodified stem cells.

7. The method of claim 5, further comprising inducing a subsequent expression of NKX6.1.

8. The method of claim 5, wherein said stem cells are autologous stem cells.

9. The method of claim 5, wherein said stem cells are autologous adipose derived stem cells.

10. The method of claim 5, wherein said stem cells are adipose tissue derived stem cells.

11. The method of claim 5, wherein a medium comprising B18R protein is used in step b).

12. The method of claim 5, wherein PDX1, NGN3, and MAFA are each expressed for 1 to 6 days before initiating a next protein.

13. The method of claim 5, wherein PDX1, NGN3, and MAFA are each expressed for about 3 days before initiating a next protein.

14. A composition comprising a population of induced beta-cells differentiated from adult unmodified stem cells transduced with one or more self-replicating RNAs to allow sequential upregulation of genes encoding PDX1 before NGN3, and NGN3 before MAFA, thus forming reprogrammed beta-cells able to produce insulin in response to glucose.

15. The composition of claim 14, said stem cells being adult stem cells.

16. The composition of claim 14, said stem cells being adipose derived adult stem cells.

17. The composition of claim 14, said stem cells being adipose derived adult stem cells from a patient with diabetes.

18. A method of treating diabetes, comprising introducing said composition of claim 14 into a patient.

19. The method of claim 18, comprising introducing said reprogrammed beta-cells into a pancreas of said patient.

20. A method of inducing differentiation of mammalian adult unmodified stem cells into somatic cells, the method comprising:
a) transducing a self-replicating RNA (srRNA) into the adult unmodified stem cells, said srRNA comprising (i) a 5′ cap, (ii) coding sequences of nonstructural proteins, (iii) a promoter, (iv) coding sequences of transcription factors and optionally fluorescent proteins mCherry or GFP, (v) independent ribosome entry sites (IRES), (vi) optionally a puromycin-resistance gene (Puro), and (vii) 3′ poly A tail; and
b) growing said transduced stem cells until differentiated somatic cells form;
wherein said transcription factors induce differentiation of the adult unmodified stem cells into somatic cells.

21. The method of claim 20, wherein the transcription factors are PDX1, NGN3 and MAFA, wherein the PDX1 is transduced before the NGN3 is transduced, and the NGN3 is transduced before the MAFA is transduced, and the somatic cells are beta cells.
